(19) United States
(12) Patent Application Publication
(10) Pub. No.: US 2013/0122106 A1
(43) Pub. Date: May 16, 2013

(54) DOSAGE FORM, AND METHODS OF MAKING AND USING THE SAME, TO PRODUCE IMMUNIZATION IN ANIMALS AND HUMANS

(71) Applicant: Aphios Corporation, Woburn, MA (US)

(72) Inventor: Trevor Percival CASTOR, Arlington, MA (US)

(73) Assignee: Aphios Corporation, Woburn, MA (US)

(21) Appl. No.: 13/656,175

(22) Filed: Oct. 19, 2012

Related U.S. Application Data
(60) Provisional application No. 61/549,055, filed on Oct. 19, 2011.

(51) Int. Cl. A61K 9/19; A61K 39/07; A61K 9/16

(52) U.S. Cl. CPC A61K 9/19; A61K 9/1641; A61K 39/07; A61K 9/1682
USPC 424/499; 424/190.1; 424/234.1; 424/252.1

(57) ABSTRACT

An embodiment of the present invention features a dosage form for administering antigen to cause an immune response in an animal or human subject in the nature of a vaccine. The dosage form comprises spheres having an effective amount of antigen to create an immune response and having an average diameter of 0.01 to 10.0 microns. The spheres comprise a polymer selected from the group consisting of poly(L-lactic acid), poly(D, L-lactic acid), poly(glycolic acid) and carboxylic acid and ester derivatives thereof, poly(fumaric anhydride) and poly(sebacic anhydride) and derivatives thereof. The spheres can be lyophilized and stored as a powder prior to use. The spheres can then be reconstituted and formulated in buffers with adjuvants.

### RELATED APPLICATIONS

This application is a continuation in part and claims priority to U.S. provisional patent application Ser. No. 61/549,055, filed Oct. 19, 2011, the entire contents of which is incorporated by reference herein.

### STATEMENT REGARDING FEDERAL SUPPORT

Work in support of the present invention was funded in part by Grant No. 1R43 GM57118-01 from the United States National Institute of General Medical Sciences and the National Institute of Health.

### FIELD OF THE INVENTION

Embodiments of the present invention are directed to vaccines and vaccine formulations.

### BACKGROUND OF THE INVENTION

Vaccines are an important tool for developing immunity to disease pathogens. Vaccines introduce a material associated with a disease pathogen to a subject's immune system. The immune system recognizes the material as foreign and develops an immune response to the material and the disease pathogen. The subject's immune system is prepared for the disease pathogen and can mount an appropriate defense if such pathogen is introduced to the subject. A material associated with a disease pathogen, such as a particular compound, often a protein or protein fragment, is called an antigen.

It is useful to sustain the antigen in the subject's immune system over time. Adjuvants are compounds and materials used to augment the immune response to make the immunity caused by the vaccine antigen longer lasting and stronger. Adjuvants can take several forms and are sometimes associated with the sustained release of antigen over time. Adjuvants approved for human use are limited to aluminum gels and aluminum salts.

Microencapsulation of antigens in bio-polymers has been used to release antigens over time. However, such systems have not been widely adopted. In making bio-polymers loaded with antigens, the antigens are exposed to organic solvents. Organic solvents can denature or inactivate the antigen making it less effective or non-effective. Additionally, organic solvents are potentially carried forward to the finished vaccine and are not desired due to potential adverse reactions.

The use of organic solvents in the manufacture of vaccines is also undesirable from an ecological perspective and is increasingly regulated by governments. Present vaccine manufacturing with bio-polymers is time consuming, costly and inefficient.

It would be useful to have vaccines which release a controlled sustained amount of antigens associated with a disease pathogen over time to create high levels of immunity to a disease pathogen. Vaccine formulations, which do not have high levels of organic solvents, are desirable. It is also desirable to avoid the use of organic solvents in their manufacture.

### SUMMARY OF THE INVENTION

Embodiments of the present invention feature vaccines which release a controlled sustained amount of an antigen associated with a disease pathogen over time to create high levels of immunity to a disease pathogen. The vaccine formulations of the present invention do not have high levels of organic solvents and avoid the use of organic solvents in their manufacture. One embodiment is directed to a dosage form for administering antigens to cause an immune response in an animal or human subject in the nature of a vaccine. The dosage form comprises one or more spheres having an average diameter of 0.01 to 10.0 microns. The spheres comprise a polymer selected from the group consisting of poly(L-lactic acid), poly(D, L-lactic acid), poly(glycolic acid) and carboxylic acid and ester derivatives thereof, poly(fumaric anhydride) and poly(sebacic anhydride) and derivatives thereof. The one or more spheres have an effective amount of the antigen to create an immune response in an animal or human when administered by intramuscular or subcutaneous injection to produce an immunological response conveying immunity to the pathogen to which the antigen is associated.

As used herein, the term “antigen” includes killed and/or attenuated organisms, toxoids, protein subunits derived from disease pathogens and conjugated compounds associated with disease pathogens. Embodiments of the present invention feature disease pathogens Bacillus anthracis, Yersinia pestis, and Brucella melitensis. These pathogens are associated with anthrax, plague and brucellosis, respectively. For example, without limitation, one embodiment of the present invention directed to the disease pathogen Bacillus anthracis features an antigen known as rPA. This antigen is a recombinant protein subunit of an antigenic protein derived from the pathogen.

One embodiment of the present invention features a polymer further comprising polyvinyl alcohol. The polyvinyl alcohol can be distributed throughout the sphere or may vary in concentration. For example, without limitation, the spheres have an interior mass and an exterior surface. The polyvinyl alcohol, in one embodiment has a distribution between the interior mass and exterior surface that is higher towards the exterior surface. A further embodiment of the present invention is directed to a method of immunizing an animal or human. The method comprises the step of providing a dosage form comprising one or more spheres having an average diameter of 0.01 to 10.0 microns. The spheres comprise a polymer selected from the group consisting of poly(L-lactic acid), poly(D, L-lactic acid), poly(glycolic acid) and carboxylic acid and ester derivatives thereof, poly(fumaric anhydride) and poly(sebacic anhydride) and derivatives thereof. The one or more spheres have an effective amount of antigen to produce an immunological response when the one or more spheres are administered by intramuscular or subcutaneous injection, conveying immunity to the pathogen to which the antigen is associated. The method further comprises the step of administering the dosage form to the animal or human to provide immunity to the pathogen.

Embodiments of the present invention feature disease pathogens Bacillus anthracis (anthrax), Yersinia pestis (plague), and Brucella melitensis (brucellosis). For example, without limitation, one embodiment of the present invention directed to the disease pathogen Bacillus anthracis features an antigen known as rPA.

The one embodiment of the method features a dosage form wherein the polymer further comprises polyvinyl alcohol. The polyvinyl alcohol is distributed equally throughout the sphere or is distributed in different concentrations throughout the sphere. For example, without limitation, one embodiment features one or more spheres, wherein each sphere has an interior area and an exterior surface. The polyvinyl alcohol has a distribution between the interior area and exterior surface that is higher towards the exterior surface.

A further embodiment of the present invention is directed to a method of making a dosage form for administering antigen to cause an immune response in an animal or human subject in the nature of a vaccine. The method comprises the step of forming a solution or nano-particle suspension of a polymer selected from the group consisting of poly(L-lactic acid), poly(D, L-lactic acid), poly(glycolic acid) and carboxylic acid and ester derivatives thereof, poly(fumaric anhydride) and poly(sebacic anhydride) and derivatives thereof, and an antigen derived from a pathogen to which immunity is desired in a supercritical, critical or near critical fluid. Next, the solution of nano-particle suspension is decompressed in a decompression fluid selected from the group consisting of water, liquid nitrogen and low pressure atmosphere. Upon decompression, one or more spheres having an average diameter of 0.01 to 10.0 microns are formed which one or more spheres have an effective amount of the antigen for creating an immune response upon intramuscular or subcutaneous administration.

Embodiments of the present invention feature a supercritical, critical or near critical fluid. A material becomes a critical fluid at conditions which equal its critical temperature and critical pressure. A material becomes a supercritical fluid at conditions which equal or exceed both its critical temperature and critical pressure. The parameters of critical temperature and critical pressure are intrinsic thermodynamic properties of all sufficiently stable pure compounds and mixtures. Carbon dioxide, for example, becomes a supercritical fluid at conditions which equal or exceed its critical temperature of 31.1° C. and its critical pressure of 72.8 atm (1,070 psig). In the supercritical fluid region, normally gaseous substances such as carbon dioxide become dense phase fluids which have been observed to exhibit greatly enhanced solvating power. At a pressure of 3,000 psig (204 atm) and a temperature of 40° C., carbon dioxide has a density of approximately 0.8 g/cc and behaves much like a nonpolar organic solvent, having a dipole moment of zero Debyes.

A supercritical fluid displays a wide spectrum of solvation power as its density is strongly dependent upon temperature and pressure. Temperature changes of tens of degrees or pressure changes by tens of atmospheres can change a compound's solubility in a supercritical fluid by an order of magnitude or more. This feature allows for the fine-tuning of solvation power and the fractionation of mixed solutes. The selectivity of nonpolar supercritical fluid solvents can also be enhanced by addition of compounds known as modifiers (also referred to as entrainers or cosolvents). These modifiers are typically somewhat polar organic solvents such as acetone, ethanol, methanol, methylene chloride or ethyl acetate. Varying the proportion of modifier allows wide latitude in the variation of solvent power.

In addition to their unique solubilization characteristics, supercritical fluids possess other physicochemical properties which add to their attractiveness as solvents. They can exhibit liquid-like density yet still retain gas-like properties of high diffusivity and low viscosity. The latter increases mass transfer rates, significantly reducing processing times. Additionally, the ultra-low surface tension of supercritical fluids allows facile penetration into microporous materials, increasing extraction efficiency and overall yields.

A material at conditions that border its supercritical state will have properties that are similar to those of the substance in the supercritical state. These so-called “near-critical” fluids are also useful for the practice of this invention. For the purposes of this invention, a near-critical fluid is defined as a fluid which is (a) at a temperature between its critical temperature (T_{c}) and 75% of its critical temperature and at a pressure at least 75% of its critical pressure, or (b) at a pressure between its critical pressure (P_{c}) and 75% of its critical pressure and at a temperature at least 75% of its critical temperature. In this definition, pressure and temperature are defined on absolute scales, e.g., Kelvin and psia. To simplify the terminology, materials which are utilized under conditions which are supercritical, near-critical, or exactly at their critical point will jointly be referred to as “SCCNC” fluids or referred to as “SFS.”

Embodiments of the present invention feature supercritical, critical or near critical fluids selected from the group of gases comprising carbon dioxide, propane, flouro-hydrocarbons, nitrous oxide, ethylene, and ethane. These gases are not considered organic solvents even though, with the exception of nitrous oxide, having a carbon component because they are gases at room temperature and pressure and are not thought to exist in the final product in concentrations greater than normal atmospheric concentrations. Although modifiers are used with supercritical critical and near critical fluids, embodiments of the present invention do not feature the use of modifiers such as methylene chloride, acetone or methanol. These modifiers can have adverse medical reactions in the subjects exposed to them.

Embodiments of the present invention feature a polymer further comprising polyvinyl alcohol. Polyvinyl alcohol is incorporated uniformly throughout each sphere, or has a distribution in each sphere. One method of the present invention features the presence of polyvinyl alcohol in the decompression fluid. The presence of the polyvinyl alcohol in the decompression fluid creates a higher concentration of polyvinyl alcohol about the surface of the sphere.

Embodiments of the present invention feature disease pathogens Bacillus anthracis (anthrax), Yersinia pestis (plague), and Brucella melitensis (brucellosis). For example, without limitation, one embodiment of the present invention directed to the disease pathogen Bacillus anthracis features an antigen known as rPA.

Embodiments of the present invention avoid organic solvents such as methylene chloride. Organic solvents are associated with adverse reactions for subjects receiving medicaments with such compounds and individuals participating in manufacturing processes which utilize such. Organic solvents raise special environmental issues particularly when employed in large scale manufacturing processes.

These and other features and advantages will be apparent to those skilled in the art upon viewing the drawings and reading the detailed description that follows.

### BRIEF DESCRIPTION OF THE DRAWINGS

FIG. 1 depicts a sphere having features of the present invention.

FIG. 2 depicts an apparatus for making a dosage form having features of the present invention.

### DETAILED DESCRIPTION OF THE INVENTION

Embodiments of the present invention will be described in detail with respect to disease pathogens Bacillus anthracis, Yersinia pestis, and Brucella melitensis with the understanding that other disease pathogens for which antigens exist or can be readily identified can be used as well. Those disease pathogens for which an antigen or attenuated pathogen has not been identified can be used as a killed pathogen. This detailed description is directed to the best mode or modes to practice the invention as presently contemplated. However, these best modes may change over time and should not be considered limiting. The vaccine formulations of the present invention do not have high levels of organic solvents and avoid the use of organic solvents in their manufacture. The formulations release a controlled sustained amount of antigen associated with a disease pathogen over time to create high levels of immunity to a disease pathogen.

The present discussion features recombinant protective antigen or rPA. See: Flick-Smith, H. C., Walker, N. J., Gibson, P., Bullifent, H., Hayward, S., Miller, J., Titball, R. W., Williamson, E. D. (2002) A Recombinant Carboxy-Terminal Domain of the Protective Antigen of Bacillus anthracis Protects Mice against Anthrax Infection. Infect. Immun. 70, 1653-1656.

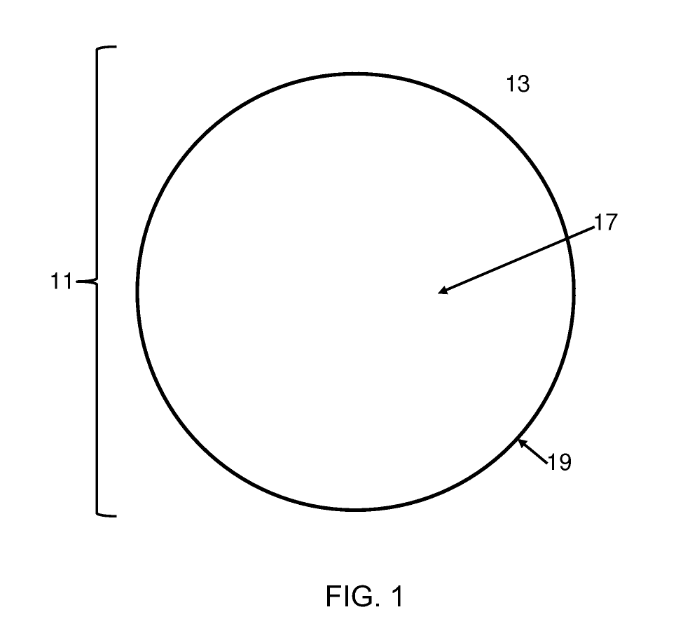

Turning now to FIG. 1, a dosage form, for administering antigen to cause an immune response in an animal or human subject in the nature of a vaccine, embodying features of the present invention, generally designated by the numeral 11, is depicted. The dosage form comprises one or more spheres 13 having an average diameter of 0.01 to 10.0 microns. The spheres 13 comprise a polymer selected from the group consisting of poly(L-lactic acid), poly(D, L-lactic acid), poly(glycolic acid) and carboxylic acid and ester derivatives thereof, poly(fumaric anhydride) and poly(sebacic anhydride) and derivatives thereof. Such polymers are available from several vendors. For example, without limitation, poly(D,L-lactide-co-glycolide) polymer in a 50:50 ratio of monomers is available from Boehringer Ingelheim KG under the trademark, RESOMER® and under other ratios from Alkermes, Inc (Cincinnati, Ohio) under the trademark, MEDISORB®.

The one or more spheres have an effective amount of antigen, rPA, to create an immune response in an animal or human when administered by intramuscular or subcutaneous injection to produce an immunological response conveying immunity to the pathogen from which the antigen is associated with. Normally, a plurality of spheres 13 are suspended in normal saline with suitable preservatives in a vial [not shown]. The spheres 13 may be lyophilized for later reconstitution. The vial may be combined with injection needles or other administration tools known in the art as part of an immunization kit.

Referring again to FIG. 1, the sphere 13 has an interior area 17 and an exterior surface 19. One embodiment of the present invention features a polymer having polyvinyl alcohol. Polyvinyl alcohol is a common polymer and is available from numerous vendors. The polyvinyl alcohol is distributed throughout the sphere 13 or is distributed in different concentrations. For example, in one embodiment the polyvinyl alcohol has a distribution between the interior area 17 and the exterior surface 19 such that the concentration is higher towards the exterior surface.

The use of the present invention will now be described with respect to a method of immunizing an animal or human. The method comprises the step of providing a dosage form 11 comprising one or more spheres 13 having an average diameter of 0.01 to 10.0 microns. The spheres 13 comprise a polymer selected from the group consisting of poly(L-lactic acid), poly(D, L-lactic acid), poly(glycolic acid) and carboxylic acid and ester derivatives thereof, poly(fumaric anhydride) and poly(sebacic anhydride) and derivatives thereof. The one or more spheres have an effective amount of antigen to produce an immunological response when the one or more spheres are administered by intramuscular or subcutaneous injection, conveying immunity to the pathogen to which the antigen is associated. The method further comprises the step of administering the dosage form 11 to the animal or human to provide immunity to the pathogen.

The encapsulation of the antigen in the polymer allows the biodegradable polymer to act as a controlled sustained antigen releasing system. The controlled release of antigen reduces the number of immunizing doses required to elicit a protective immune response. The polymer further protects the antigen from the actions of proteases. And, the polymer stabilizes the antigen for storage at room temperature. Product stabilization is also achieved by lyophilization of the vaccine prep unto a dry powder.

Embodiments of the present invention which have polyvinyl alcohol as an additional polymer are administered in the same way.

A further embodiment of the present invention is directed to a method of making a dosage form for administering antigen to cause an immune response in an animal or human subject in the nature of a vaccine. The method comprises the step of forming a solution or nano-particle suspension of a polymer selected from the group consisting of poly(L-lactic acid), poly(D, L-lactic acid), poly(glycolic acid) and carboxylic acid and ester derivatives thereof, poly(fumaric anhydride) and poly(sebacic anhydride) and derivatives thereof, and an antigen associated a pathogen to which immunity is desired in a supercritical, critical or near critical fluid. Next, the solution of nano-particle suspension is decompressed in a decompression fluid selected from the group consisting of water, liquid nitrogen and low pressure atmosphere. Upon decompression, one or more spheres having an average diameter of 0.01 to 10.0 microns are formed which one or more spheres have an effective amount of antigen for creating an immune response upon intramuscular or subcutaneous administration.

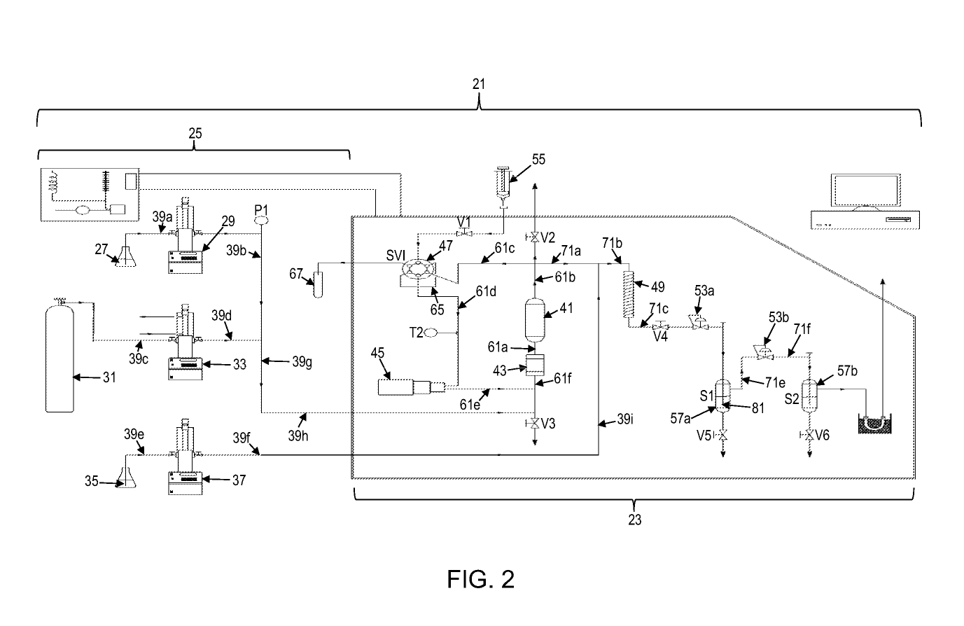

An apparatus, generally designated by the numeral 21, for performing the method of the present invention is depicted in FIG. 2. The apparatus 21 has a closed chamber 23 and an external support area 25. The closed chamber 23 is maintained at a controlled temperature. The external support area 25 has the following major elements: a co-solvent source 27, co-solvent syringe pump 29, critical fluid source 31, critical fluid syringe pump 33, feed source 35, and feed source syringe pump 37. These major elements of the external support area 25 are plumbed by conduits 39a-j to other components within the closed chamber 23 as will be described below.

The closed chamber 23 has a mixing chamber 41, a solids chamber 43, a high pressure circulation pump 45, a multi-port sampling valve 47, a static in-line mixer 49, two back pressure regulators (BPR) 53a and 53b, at least one injector 55 and two sample collection chambers 57a and 57b.

Co-solvent syringe pump 29 and critical fluid syringe pump 33 are in fluid communication with their respective sources, co-solvent source 27 and critical fluid source 31, and solids chamber 43. Solids chamber 43 is for containing polymer selected from the group consisting of poly(L-lactic acid), poly(D, L-lactic acid), poly(glycolic acid) and carboxylic acid and ester derivatives thereof, poly(fumaric anhydride) and poly(sebacic anhydride) and derivatives thereof.

The solids chamber 43 is in fluid communication with mixing chamber 41 and with the sampling valve 47 and circulation pump 45 via conduits 61a-f forming high-pressure circulation loop. Polymer is solubilized and mixed by circulation within the high-pressure circulation loop.

Sampling valve 47 is plumbed with a sampling loop 65 and can remove the sample trapped in the sampling loop 65 via sample collector 67. Solvent injector 55 permits flushing of the sampling loop 65.

A take-off conduit 71a is in fluid communication with the high pressure circulation loop at conduit 61b and 61c. Thus, dissolved polymer can exit the high-pressure circulation loop. Antigen associated with a pathogen to which immunity is desired is held in feed source 35 and pumped by the feed syringe 37 through conduits 39f and 39i in communication with take-off conduit 71a. The antigen is combined with the polymer stream and flows via conduit 71b to static mixer 49. Static mixer 49 combines and mixes the polymer and antigen.

Pressure within the system is maintained by back pressure regulators 53a and 53b. Back pressure regulator 53a is plumbed to the static mixer 49 via conduit 71c and is in further communication with first collection chamber 57a. The first collection chamber 57a contains a decompression fluid selected from the group consisting of water, liquid nitrogen and low pressure atmosphere. Upon decompression, through a nozzle 81a one or more spheres having an average diameter of 0.01 to 10.0 microns are formed. The nozzle 81a features a 10-mil (internal diameter of 0.25 mm or 250 micron) capillary. The spheres have an effective amount of antigen for creating an immune response upon intramuscular or subcutaneous administration.

When polyvinyl alcohol is desired, it is combined with the polymers in the solids chamber or placed in solution in the decompression fluid held in the first collection chamber 57a.

Second collection chamber 57b is in fluid communication with first collection chamber 57a via conduits 71e and 71f to provide extra capacity.

The operation of the apparatus 21 described above is further exemplified in the examples below.

### Example 1

### rPA-01

Spheres were formed which contained the antigen rPA in the following manner. A feed rate of 1.5 mg/ml rPA in phosphate buffer solution and a supercritical, critical or near critical solution of solution of poly(D, L-lactic acid), poly(glycolic acid) in propane was injected into a decompression fluid of 1% polyvinyl alcohol and produced a batch of spheres having a mean particle diameter of 1.54 microns. The polymer solution was maintained prior to injection at a pressure of 21 MPa and 30 degrees centigrade. The batch of spheres contained approximately 5 mg rPA.

This suspension of spheres in a phosphate buffer was then lyophilized. Dried spheres were stored at five degrees centigrade until used. Prior to use, dried spheres were re-constituted and formulated into a 20% alhydrogel (v/v) in phosphate buffer solution to produce a final concentration of 200 micrograms/ml.

### Example 2

### rPA-02

Spheres were formed which contained the antigen rPA in the following manner. A feed rate of 0.25 mg/ml rPA in phosphate buffer solution and a supercritical, critical or near critical solution of solution of poly(D, L-lactic acid), poly(glycolic acid) in propane was injected into a decompression fluid of 1% polyvinyl alcohol and produced a batch of spheres having a mean particle diameter of 0.61 microns. The polymer solution was maintained prior to injection at a pressure of 21 MPa and 30 degrees centigrade. The batch of spheres contained approximately 3.6 mg rPA.

This suspension of spheres in a phosphate buffer was then lyophilized. Dried spheres were stored at five degrees centigrade until used. Prior to use, dried spheres were re-constituted and formulated into a 20% alhydrogel (v/v) in phosphate buffer solution to produce a final concentration of 200 micrograms/ml.

### Example 3

### rPA-03

Spheres were formed which contained the antigen rPA in the following manner. A feed rate of 0.25 mg/ml rPA in phosphate buffer solution and a supercritical, critical or near critical solution of solution of poly(D, L-lactic acid), poly(glycolic acid) in propane was injected into a decompression fluid of de-ionized water and produced a batch of spheres having a mean particle diameter of 0.37 microns. The polymer solution was maintained prior to injection at a pressure of 21 MPa and 30 degrees centigrade. The batch of spheres contained approximately 9.6 mg rPA.

This suspension of spheres in a phosphate buffer was then lyophilized. Dried spheres were stored at five degrees centigrade until used. Prior to use, dried spheres were re-constituted and formulated into a 20% alhydrogel (v/v) in phosphate buffer solution to produce a final concentration of 200 micrograms/ml.

### Example 4

### Control

A formulation of rPA in 20% alhydrogel in phosphate buffer solution to produce a final concentration of 200 micrograms rPA per milliliter was made.

### Example 5

Female adult AU mice were immunized once with a 20 microgram dose of rPA, 0.1 ml of the control of Example 4 and the formulation of Example 1. The response to immunization was monitored by sampling mice at day fourteen and measuring IgG titers to rPA in serum samples by standard ELISA.

Mice were challenged on day 21 with 10³ MLD (10⁶ cfu) Bacillus anthracis STI strain intraperitoneally. Survival was observed over subsequent fourteen days. The control group receiving the formulation of Example 4 exhibited a survival rate of 100% at day 21 and geometric mean titers of IgG of 0.5.

The group receiving spheres containing rPA made in accordance with Example 1 exhibited a survival rate of 100% at day 21 and a geometric mean titer of IgG of 0.61 suggesting a stronger immune response to the immunization than rPA in alhydrogel without encapsulation in spheres.

Mice which were not immunized exhibited a survival rate of 0%. That is, there were no survivors.

Thus, the inventions have been described in detail with respect to the best mode. Those skilled in the art will readily understand that the description is capable of modification and alteration without departing from the teaching herein. Therefore, the invention should not be limited to the precise details presented but should encompass the subject matter of the claims that follow and their equivalents.

1. A dosage form for administering antigen to cause an immune response in an animal or human subject in the nature of a vaccine comprising:
one or more spheres having an average diameter of 0.01 to 10.0 microns, said spheres comprising a polymer selected from the group consisting of poly(L-lactic acid), poly(D, L-lactic acid), poly(glycolic acid) and carboxylic acid and ester derivatives thereof, poly(fumaric anhydride) and poly(sebacic anhydride) and derivatives thereof, and said one or more spheres having an effective amount of antigen associated with a disease pathogen,
said one or more spheres for administration by intramuscular or subcutaneous injection to produce an immunological response conveying immunity to the pathogen to which the antigen is associated.

2. The dosage form of claim 1 wherein said pathogen is Bacillus anthracis.

3. The dosage form of claim 2 wherein said antigen is rPA.

4. The dosage form of claim 1 wherein said polymer further comprises polyvinyl alcohol.

5. The dosage form of claim 6 wherein said spheres have an interior mass and an exterior surface, said polyvinyl alcohol having a distribution between said interior mass and exterior surface that is higher towards said exterior surface.

6. The dosage form of claim 1 wherein said antigen is derived from the pathogen Yersinia pestis.

7. The dosage form of claim 1 wherein said antigen is derived from the pathogen Brucella melitensis.

8. A method of immunization of an animal or human comprising the steps of:
providing a dosage form comprising one or more spheres having an average diameter of 0.01 to 10.0 microns, said spheres comprising a polymer selected from the group consisting of poly(L-lactic acid), poly(D, L-lactic acid), poly(glycolic acid) and carboxylic acid and ester derivatives thereof, poly(fumaric anhydride) and poly(sebacic anhydride) and derivatives thereof, and said one or more spheres having an effective amount of antigen to a disease pathogen to which the antigen is associated, said one or more spheres for administration by intramuscular or subcutaneous injection to produce an immunological response conveying immunity to the pathogen to which the antigen is associated; and,
administering said dosage form to said animal or human to provide immunity to said pathogen.

9. The method of claim 8 wherein said pathogen is Bacillus anthracis.

10. The method of claim 9 wherein said antigen is rPA.

11. The method of claim 8 wherein said polymer further comprises polyvinyl alcohol.

12. The method of claim 11 wherein said spheres have an interior mass and an exterior surface, said polyvinyl alcohol having a distribution between said interior mass and exterior surface that is higher towards said exterior surface.

13. The method of claim 8 wherein said antigen is derived from the pathogen Yersinia pestis.

14. The method of claim 8 wherein said antigen is derived from the pathogen Brucella melitensis.

15. A method of making a dosage form for administering antigen to cause an immune response in an animal or human subject in the nature of a vaccine, comprising steps of: forming a solution or nano-particle suspension of a polymer selected from the group consisting of poly(L-lactic acid), poly(D, L-lactic acid), poly(glycolic acid) and carboxylic acid and ester derivatives thereof, poly(fumaric anhydride) and poly(sebacic anhydride) and derivatives thereof, and an antigen associated with a pathogen to which immunity is desired in a supercritical, critical or near critical fluid and decompressing the solution or suspension in a decompression fluid selected from the group consisting of water, liquid nitrogen and low pressure atmosphere to form one or more spheres having an average diameter of 0.01 to 10.0 microns and having an effective amount of antigen for creating a an immune response upon intramuscular or subcutaneous administration

16. The method of claim 15 wherein said pathogen is Bacillus anthracis.

17. The method of claim 16 wherein said antigen is rPA.

18. The method of claim 15 wherein said polymer further comprises polyvinyl alcohol.

19. The method of claim 18 wherein said decompression fluid comprised polyvinyl alcohol.

20. The method of claim 15 wherein said antigen is derived from the pathogen Yersinia pestis.

21. The method of claim 15 wherein said antigen is derived from the pathogen Brucella melitensis.

22. The method of claim 15 wherein the suspension of spheres is lyophilized to produce a dry powder for improving the shelf stability of the vaccine product.

23. The method of claim 22 wherein the dry powder is reconstituted by formulation in appropriate biological buffers with vaccine adjuvants such as 20% alhydrogel (v/v).
